(19) United States
(12) Patent Application Publication
(10) Pub. No.: US 2016/0346189 A1
(43) Pub. Date: Dec. 1, 2016

(54) Multi-Component Products and Multi-Step Regimens For Improving The Appearance Or Feel Of Human Skin

(71) Applicant: The Procter & Gamble Company, Cincinnati, OH (US)

(72) Inventors: Joseph Harry JANSEN, Harrison, OH (US); Joseph Michael ZUKOWSKI, Cincinnati, OH (US); Paul Robert TANNER, Lebanon, OH (US)

(21) Appl. No.: 14/723,829

(22) Filed: May 28, 2015

(51) Int. Cl. A61K 8/891; A61K 8/73; A61Q 19/02; A61Q 17/04; A61Q 19/00; A61Q 19/08; A61K 8/34; A61K 8/02

(52) U.S. Cl. CPC A61K 8/891; A61K 8/345; A61K 8/732; A61K 8/025; A61Q 17/04; A61Q 19/007; A61Q 19/08; A61Q 19/02; A61K 2800/884

(57) ABSTRACT

Described are multi-component products and multi-step regimens for improving the look and/or feel of human skin. The products comprise a first composition comprising a humectant, a second composition comprising a non-volatile oil, and a third composition comprising substantially spherical particles. The regimens comprise the steps of: applying to the skin a first layer formed using a first composition comprising a humectant, followed by applying a second layer formed using a second composition comprising at least one non-volatile oil, followed by applying a third layer formed using a third composition comprising substantially spherical particles.

### FIELD OF THE INVENTION

The present invention relates to a multi-component product and a multi-step method for improving the appearance or feel of human skin. The method comprises applying to the skin a first composition that comprises at least one humectant, thereafter applying a second composition that comprises a non-volatile oil, and thereafter applying a third composition that is water-based and comprises a high concentration of particulate material. The multi-component product comprises the first, second and third compositions.

### BACKGROUND OF THE INVENTION

Personal care products are well known and widely used. These products have long been employed to cleanse and moisturize, deliver actives, hide imperfections and to reduce the oiliness and shine on keratinous surfaces. Personal care products have also been used to alter the color and appearance of skin and hair. A variety of personal-care compositions are available to provide skin care benefits and to counteract what many consider undesirable “signs of skin aging,” such as fine lines, wrinkles, and uneven skin texture. Of these benefits, the look and feel of human skin are arguably the two most important and desired effects by consumers.

Many products are designed to improve the look of human skin and many products are directed to improving the feel. Traditionally, a wide variety of different functional materials are combined in a single skin care product in an attempt to deliver a range of benefits to consumers. For example, a typical skin care product might contain: humectants and other skin actives to improve the condition and health of the skin; emollients to lubricate the skin; and a wide variety of powders to provide a skin feel and immediate skin appearance benefit. But combining compositions into one product often has difficulties.

Moreover, products that deliver one benefit are generally intended as a single application product. Layering many products on the skin that deliver different benefits, but are intended to be used individually, may have the same drawbacks and complications as mixing too many ingredients in one composition. Hence there is a need for products and regimens that deliver multiple benefits, in different compositions, but are designed to be used together in a coordinated regimen.

For example, particulate material can be added to consumer products for a variety of reasons such as to improve the skin feel of the product. Particulate materials also may provide an immediate visible benefit to the skin by diffusely reflecting light, which provides a matting effect to the skin. However, many particulate materials are added to act as an opacifying agent, which effectively turns the consumer product into make-up or a make-up like product. The acute and chronic benefits of personal care compositions having opacifying agents are often lost on the user who only appreciates the masking effect the opacifying agents provide. Examples of these include high refractive index pigments, such as titanium dioxide and iron oxides, to provide skin color benefits.

Micronized or spherical polymer particles are used to provide feel and visible texture, wrinkle reduction benefits. For these materials, however, there are tradeoffs if one attempts to increase these feel and look benefits. Using high levels of powder typically lead to products that are hard to spread on skin, and that lose their look benefits over time. These products typically become noticeably white and can flake off the skin.

Likewise, humectants provide a multitude of skin health and appearance benefits, such as: increasing skin translucency, as evidenced by less surface scattering and reducing refractive index gradients in the stratum corneum; reducing visible texture, that is, plumping of the stratum corneum; and generally better functioning and stronger skin. Glycerin is the most efficient humectant available due to its chemical structure. But glycerin is a very thick, sticky material and high levels of glycerin can feel very sticky and heavy on the skin. Moreover, high levels of glycerin on the skin can make it look very shiny and greasy, given that glycerin is slow to absorb into the skin.

The above issues can be addressed by delivering personal care compositions with a high percentage of a particulate material having a low refractive index that can be applied on top of one or more personal care compositions that contain high levels of humectants. See U.S. Provisional Application No. 61/927,255. This layering effect avoids the drawbacks of combining an “all-in-one” composition, and the drawbacks of layering products not designed to be used together. That is, separating a product in a specific way into multiple layers and applying those layers to skin in a specific sequence, skin feel and skin appearance benefits can be enhanced.

Although the compositions and regimens described in U.S. Provisional Application No. 61/927,255 provide numerous benefits over the prior art, Applicants have discovered that compositions and regimens that provide a separate non-volatile oil containing composition between the humectants/skin active layer and the powder layer provide benefits as well. Without being bound by theory, it is believed that further separating the humectant(s) materials from the particulate material(s), by use of a separate intervening non-volatile oil layer, further enhances feel and/or appearance benefits.

### SUMMARY OF THE INVENTION

There is provided a multi-component product comprising;
a. a first composition comprising at least one humectant;
b. a second composition comprising a non-volatile oil; and
c. a third composition comprising from about 10% to about 30%, by weight of the third composition, of substantially spherical particles selected from the group consisting of coated starch, uncoated starch, coated starch derivatives, uncoated starch derivatives, coated crosslinked starch, uncoated crosslinked starch, coated silicone elastomer particles, uncoated silicone elastomer particles, and combinations thereof.

There is also provided a method of improving the look and/or feel of human skin comprising the steps of: applying to the skin a first layer formed by using a first composition comprising at least one humectant; thereafter applying a second layer formed using a second composition comprising at least one non-volatile oil; and thereafter applying a third layer formed using a third composition comprising from about 10% to about 30%, by weight of the third composition, of substantially spherical particles selected from the group consisting of coated starch, uncoated starch, coated starch derivatives, uncoated starch derivatives, coated crosslinked starch, uncoated crosslinked starch, coated silicone elastomer particles, uncoated silicone elastomer particles, and combinations thereof.

The methods, regimens and products of the present invention overcome many issues of prior personal care compositions and regimens. By placing different components in different compositions and applying them in order in a multi step process, the sticky, heavy feel of the humectants is greatly reduced, the smooth, powdery feel of the particles is enhanced, and the appearance from the particles is increased.

### DETAILED DESCRIPTION

The multi-component products and multi-step regimens of the present invention may be used in skin care and cosmetic products, non-limiting uses of which include moisturizers, conditioners, anti-aging compounds, skin lightening compounds, UV actives, and combinations thereof. The products and regimens are designed to improve the look, feel, or both of keratinous tissue of the face, neck, hands, arms and other areas of the body. It will be understood that it is possible to apply additional compositions or layers immediately before or immediately after applying the first, second or third compositions described herein without departing from the spirit and scope of the present invention.

Percentages are by weight of the personal care composition or the particular phase being described, unless otherwise specified. All ratios are weight ratios, unless specifically stated otherwise. All numeric ranges are inclusive of narrower ranges. The number of significant digits conveys neither limitation on the indicated amounts nor on the accuracy of the measurements. All measurements are understood to be made at ambient conditions, where “ambient conditions” means conditions at about 25° C., under about one atmosphere of pressure, and at about 50% relative humidity.

### DEFINITIONS

“Apply” or “application,” as used in reference to a composition, means to apply or spread the composition onto a keratinous tissue surface.

“Derivative” refers to a molecule similar to that of another one, but differing from it in respect of a certain functional moiety. Derivatives may be formed by known reactive pathways. Suitable functional moieties include esters, ethers, amides, amines, carboxylic acids, hydroxyls, halogens, thiols, and/or salt derivatives of the relevant molecule.

“Keratinous tissue” refers to keratin-containing layers disposed as the outermost protective covering of mammals which includes, but is not limited to, skin, hair, nails, cuticles, etc.

“Non-volatile” means a material that exhibits a vapor pressure of no more than about 0.2 mm Hg at 25° C. at one atmosphere and/or a material that has a boiling point at one atmosphere of at least about 300° C. “Volatile” means that the material exhibits a vapor pressure of greater than about 0.2 mm Hg at 25° C.

“Personal care product” means a product with a composition suitable for topical application on mammalian keratinous tissue.

“Regulating skin condition” means improving skin appearance and/or feel, for example, by providing a benefit, such as a smoother appearance and/or feel. Herein, “improving skin condition” means effecting a visually and/or tactilely perceptible positive change in skin appearance and/or feel. The benefit may be a chronic or acute benefit and may include one or more of the following: reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

“Safe and effective amount” means an amount of a compound or composition sufficient to induce a positive benefit but low enough to avoid serious side effects (i.e., provides a reasonable benefit to risk ratio within the judgment of a skilled artisan).

“Signs of skin aging,” include, but are not limited to, all outward visibly and tactilely perceptible manifestations, as well as any macro- or micro-effects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

“Substantially free of” as used herein, means that the composition comprises less than about 1%, more preferably less than about 0.5%, even more preferably less than about 0.25%, and most preferably less than about 0.1%, by weight of the composition, of the stated ingredient.

“Substituted” means comprising at least one heteroatomic substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups.

### Methods of Using the Multi-Component Products

In one aspect, the present invention concerns a method of providing benefits to human skin, specifically, the benefits of improving the look or feel of human skin. Even more specifically, the described compositions may be applied to skin exhibiting signs of skin aging, for example, to reduce the appearance of skin texture issues, which include reducing the appearance of wrinkles, reducing the appearance of deep lines, reducing the appearance of fine lines, reducing the appearance of large pores and bumps on the skin. Further the skin is hydrated and moisturized. The individual ingredients of the compositions of this invention, both required and optional, as well as their properties and concentration levels, are defined in greater detail below.

There is provided herein a multi-step regimen where the first step is applying a first layer using a first composition comprising one or more humectants. The second step is applying a second layer using a second composition comprising a non-volatile oil. The third step is applying a third composition comprising particulate material.

The first, second and third compositions are kept separate until use to insure they can be applied in the proper order, which, as the comparative examples will illustrate, is an important aspect of the invention. The compositions can, however, be sold in a variety of ways. The multiple compositions can be sold in the same package as a multi-component product, if separated in the package and when dispensed. The compositions can be sold in individual containers that are sold separately or together (again, as a multi-component product). Non-limiting examples include one box that contains multiple bottles, each bottle containing a different layer, or the layers can be sold separately in an array of packages where the consumer can select from various different versions of each layer.

The compositions may be applied by a variety of means, including by rubbing, wiping or dabbing with hands or fingers, or by means of an implement and/or delivery enhancement device. Non-limiting examples of implements include a sponge or sponge-tipped applicator, a swab (for example, a cotton-tipped swab), a pen optionally comprising a foam or sponge applicator, a brush, a wipe, and combinations thereof. Non-limiting examples of delivery enhancement devices include mechanical, electrical, ultrasonic and/or other energy devices. After application, the composition may be allowed to remain on the skin.

The amount of the compositions applied, the frequency of application and the period of use will vary widely depending upon the level of components of a given composition and the level of regulation desired. For example, from about 0.1 mg composition/cm² to about 50 mg composition/cm², and alternatively about 2 mg composition/cm² of composition may be applied. In one embodiment, the compositions are applied at least once daily, where “daily” and “days” mean a 24-hour period. The user may be instructed to reapply the composition(s) after a period of time has passed, for example every hour, and alternatively when the composition(s) has been washed or rinsed from the skin, for example after washing one's hands or face, or after swimming, bathing or showering. The composition(s) may be applied as part of a treatment regimen, for example, once daily for 30 consecutive days, alternatively for 14 consecutive days, alternatively for 7 consecutive days and alternatively for 2 consecutive days.

### Personal-Care Compositions for Use in a Multi-Step Regimen

The compositions used in the products and regimens of the present invention, which are described in more detail below, are useful for improving skin appearance and/or feel. The compositions may be useful for regulating skin condition and improving skin condition. In certain embodiments, the composition is useful for regulating and improving the signs of skin aging. The compositions may provide an essentially immediate (i.e., acute) improvement in skin appearance and/or feel following application. It is believed that the acute improvement may be attained with a single or limited number of applications of the composition. However, the compositions may comprise components that provide a gradual (i.e., chronic) improvement. It is believed that the chronic improvement may involve multiple, reoccurring, or periodic applications of the composition. The compositions of the present invention may be incorporated into consumer products. In certain embodiments, the consumer products allow the composition to be applied as a spot treatment over a limited area of the skin. In one embodiment, the compositions of the present invention yield a visibly noticeable reduction in wrinkles or bumps on the skin.

Each of the first composition, the second composition and the third composition typically comprises water. The aqueous phase of one or more of the first, second or third compositions may comprise components other than water (non-water components), including but not limited to water-soluble moisturizing agents, conditioning agents, anti-microbials, humectants and other water-soluble skin care actives, to impart an increased benefit to the keratinous tissue.

### First Composition

The first composition of the present invention includes one or more humectants. The first composition may comprise from about 3% to about 30%, alternatively, from about 5% to about 25%, or from about 10% to about 20%, by weight of the first composition, of the humectant.

An exemplary class of humectants is polyhydric alcohols. Suitable polyhydric alcohols include polyalkylene glycols and alkylene polyols and their derivatives, including propylene glycol, dipropylene glycol, polypropylene glycol, polyethylene glycol and derivatives thereof; sorbitol; hydroxypropyl sorbitol; erythritol; threitol; pentaerythritol; xylitol; glucitol; mannitol; butylene glycol (e.g., 1,3-butylene glycol); pentylene glycol; hexane triol (e.g., 1,2,6-hexanetriol); glycerin; ethoxylated glycerine; and propoxylated glycerine.

Other suitable humectants include sodium 2-pyrrolidone-5-carboxylate; guanidine; glycolic acid and glycolate salts (e.g., ammonium and quaternary alkyl ammonium); lactic acid and lactate salts (e.g., ammonium and quaternary alkyl ammonium); aloe vera in any of its variety of forms (e.g., aloe vera gel); hyaluronic acid and derivatives thereof (e.g., salt derivatives such as sodium hyaluronate); lactamide monoethanolamine; acetamide monoethanolamine; urea; sodium pyroglutamate, water-soluble glyceryl poly(meth)acrylate lubricants (such as Hispagel®) and mixtures thereof.

Glycerin is a preferred humectant and can be the sole humectant used. Preferably, the humectant component comprises at least about 50%, more preferably, at least about 75%, by weight, glycerin.

The first composition can be in any of a variety of forms, non-limiting examples including liquids, creams and lotions. In one embodiment, the first composition is water based, preferably, a water continuous formation. In certain embodiments, the first composition can be in the form of an aqueous solution, aqueous gel, aqueous dispersion, oil-in-water emulsion, or a water-in-oil-in-water emulsion.

The first composition may comprise about 20% to about 99%, preferably about 40% to about 90%, and more preferably from about 50% to about 85%, of water.

The first composition may comprise one or more substantially spherical particulate materials, but at lower levels relative to the third composition. In a preferred embodiment, the first layer will be substantially free of substantially spherical particles. The first composition may also comprise volatile oils, non-volatile oils, or both. When non-volatile oils are present, they are preferably at lower levels than in the second composition. In a preferred embodiment, the first composition is substantially free of non-volatile oils.

### Second Composition

The multi-component products and regimens utilize a separate composition (the “second composition”) comprising at least one non-volatile oil. The amount of the non-volatile oil in the second composition is influenced by the amount of substantially spherical particles included in the third composition, described below. In one embodiment, the weight ratio of non-volatile oil in the second composition to substantially spherical particulate material in the third composition is from about 1:10 to about 3:2. In another embodiment, the weight ratio of non-volatile oil in the second composition to substantially spherical particulate material in the third composition is from about 1:4 to 1:1 and in yet another embodiment the ratio is from about 1:4 to about 3:4.

Suitable non-volatile oils include hydrocarbons, esters, amides, ethers, silicones, and mixtures thereof. Suitable non-volatile hydrocarbon oils include straight, branched, or cyclic alkanes and alkenes. The chain length may be selected based on desired functional characteristics such as viscosity. Suitable non-volatile esters include esters with hydrocarbyl chains derived from fatty acids or alcohols (e.g., mono-esters, polyhydric alcohol esters, and di- and tri-carboxylic acid esters). The hydrocarbyl radicals of the esters hereof may include or have covalently bonded thereto other compatible functionalities, such as amides and alkoxy moieties (e.g., ethoxy or ether linkages, etc.). Exemplary esters include, but are not limited to, isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, C12-15 alkyl benzoate, butyloctyl salicylate, phenylethyl benzoate, dicaprylyl carbonate, dioctyl malate, dicaprylyl maleate, isononyl isononanoate, propylene glycol dicaprate, diisopropyl adipate, dibutyl adipate, and oleyl adipate. Other suitable esters are further described in the Personal Care Product Council's International Cosmetic Ingredient Dictionary and Handbook, Thirteenth Edition, 2010, under the functional category of “Esters.”

Suitable non-volatile amides include compounds having an amide functional group while being liquid at 25° C. and insoluble in water. Suitable amides include, but are not limited to, N-acetyl-N-butylaminopropionate, isopropyl N-lauroylsarcosinate, butylphthalimide, isopropylphthalimide, and N,N,-diethyltoluamide. Other suitable amides are disclosed in U.S. Pat. No. 6,872,401.

Suitable non-volatile ethers include saturated and unsaturated fatty ethers of a polyhydric alcohol, and alkoxylated derivatives thereof. Exemplary ethers include, but are not limited to, C₄₋₂₀ alkyl ethers of polypropylene glycols, and di-C₈₋₃₀ alkyl ethers. Suitable examples of these materials include PPG-14 butyl ether, PPG-15 stearyl ether, PPG-11 stearyl ether, dioctyl ether, dodecyl octyl ether, and mixtures thereof.

Suitable non-volatile silicone oils include polysiloxanes. Non-volatile polylsiloxanes may have a viscosity of from about 10 to about 1,000,000 centistokes at 25° C. Such polysiloxanes can be represented by the general chemical formula:

R₃SiO[R₂SiO]ₓSiR₃

wherein each R is independently selected from hydrogen or C₁₋₃₀ straight or branched chain, saturated or unsaturated alkyl, phenyl or aryl, trialkylsiloxy; and x is an integer from 0 to about 10,000. In certain embodiments, R is methyl or ethyl. Commercially available polysiloxanes include the polydimethylsiloxanes, which are also known as dimethicones, examples of which include the DM-Fluid series from Shin-Etsu, the Vicasil® series sold by Momentive Performance Materials Inc., and the Dow Corning® 200 series sold by Dow Corning Corporation. Specific examples of suitable polydimethylsiloxanes include Dow Corning® 200 fluids (also sold as Xiameter® PMX-200 Silicone Fluids).

Suitable dimethicones include those represented by the chemical formula:

R₃SiO[R₂SiO]ₓ[RR′SiO]_{y}SiR₃

wherein R and R′ are each independently hydrogen or C₁₋₃₀ straight or branched chain, saturated or unsaturated alkyl, aryl, or trialkylsiloxy; and x and y are each integers of 1 to 1,000,000. Examples include alkyl dimethicones wherein at least R′ is a fatty alkyl (e.g., C₁₂₋₂₂). A suitable alkyl dimethicone is cetyl dimethicone, wherein R′ is a straight C16 chain and R is methyl, commercially available as 2502 Cosmetic Fluid from Dow Corning.

Preferred non-volatile oils include dimethicones (polydimethylsiloxanes), preferably with viscosities of between 10 cst and 1000 cst, more preferably between 15 cst to 400 cst, most preferably between 20 cst and 200 cst. The average chain lengths for these preferred dimethicone materials is from about 12 to about 375 dimethylsiloxane units, more preferably from about 20 to about 200 dimethylsiloxane units, and most preferably with average chain lengths of from about 27 to about 125 dimethylsiloxane units.

In one embodiment, the second composition will comprise at least one non-volatile silicone oil. In one such embodiment, at least about 70%, by weight of the non-volatile oil, is a non-volatile silicone oil. In another embodiment, at least about 80%, by weight of the non-volatile oil, is a non-volatile silicone. In yet another embodiment, at least about 90%, by weight of the non-volatile oil, is a non-volatile silicone oil.

The second composition can be in any of a variety of forms, non-limiting examples including liquids, creams and lotions. In one embodiment, the second composition is water based. In one embodiment, the second composition is an oil-in-water emulsion. The oil phase of the second composition, when present, may comprise a silicone oil. However, the oil phase may also comprise non-silicone oils such as hydrocarbon oils, esters, ethers, and the like.

The second composition may comprise one or more substantially spherical particles, but at low levels relative to the third composition. In a preferred embodiment, the second composition will be substantially free of substantially spherical particles. The second composition may comprise one or more humectants, but at low levels relative to the first composition. In a preferred embodiment, the second composition will be substantially free of humectants.

The second composition optionally includes at least one volatile oil. In one embodiment, the second composition comprises from about 5% to about 20%, by weight of the second composition, of a volatile oil.

### Third Composition

The third composition comprises substantially spherical particulate materials. The particulate material may be dispersed and suspended in the third composition. The particulate material may be used to provide consumer desirable look and feel properties to the composition. These particles give the composition a silky or lubricious feel which may offset the heavy greasiness associated with oils and/or the tacky feel of many humectants.

The third composition contains from about 10%, preferably from about 12%, even more preferably from about 15%, to about 30%, preferably to about 25%, by weight of the third composition, of substantially spherical particles. In one embodiment, the particles may be selected from the group consisting of coated starch, uncoated starch, coated starch derivatives, uncoated starch derivatives, coated crosslinked starch, uncoated crosslinked starch, coated silicone elastomer particles, uncoated silicone elastomer particles, and combinations thereof. Preferably the particles are selected from the group consisting of tapioca starch, corn starch, potato starch, glyceryl starch, aluminum starch octenylsuccinate, calcium starch octenylsuccinate, polymethylsilsesquioxane coated tapioca starch, cross linked starch, silicone elastomer particles, and combinations thereof.

The particulate material may exhibit a median particle size of from about 1 μm to about 40 μm; from about 2 μm to about 30 μm; from about 5 μm to about 30 μm; from about 5 μm to about 25 μm; or, alternatively, from about 5 μm to about 20 μm. Median particle size can be determined by any suitable method known in the art, such as by using coulter-counter equipment or the ASTM Designation E20-85 “Standard Practice for Particle Size Analysis of Particulate Substances in the Range of 0.2 to 75 Micrometers by Optical Microscopy,” ASTM Volume 14.02, 1993, incorporated herein by reference.

The median particle size is measured, and the concentration levels are calculated, when the particulate material is in the neat form (i.e., in the essentially pure, powder form prior to combination with the carrier of the composition).

In various embodiments, the particulates are preferably spherical. In the present invention, “spherical” and “sphere” mean not only real sphere but also include deformed sphere wherein major axis/minor axis (aspect ratio) on average is typically in the range of 1 to 2, more preferably 1 to 1.6, and even more preferably 1 to 1.4. The shape of the fine particles may be confirmed by observing the fine particle with an optical microscope or electron microscope.

The third composition can be any skin care composition, non-limiting examples include, liquids, creams and lotions. In one embodiment, the third composition is water based, more preferably, a water continuous formation. In one embodiment, the third composition is in the form of an aqueous solution, aqueous gel, aqueous dispersion, oil-in-water emulsion, or a water-in-oil-in-water emulsion.

The third composition may comprise about 20% to about 85%, preferably about 30% to about 80%, and more preferably from about 40% to about 75%, of water.

The third composition may comprise one or more non-volatile or volatile oils. When included, non-volatile oils will be at lower levels relative to the second composition. In a preferred embodiment, the third composition will be substantially free of non-volatile oils. The third composition may also comprise humectant. When humectant(s) are present, they are preferably at lower levels than in the first composition. In a preferred embodiment, the third composition is substantially free of humectant.

In one embodiment, the first, second, and third compositions comprise less than about 1%, preferably less than about 0.5%, by weight of the composition, of titanium dioxide and iron oxide particles, or other high refractive index particles (e.g., refractive index greater than about 1.6). In one such embodiment, the compositions are substantially free of titanium dioxide and iron oxide. Further, the compositions comprise less than about 4%, preferably less than about 3%, and even more preferably less than about 1%, by weight of the composition, of mica, talc, interference pigments and other substantially non-spherical particles.

At least one or a combination of the first and second compositions each have a contrast ratio of less than about 20, preferably less than about 10 and even more preferably less than about 6.

The following are representative materials for use as the substantially spherical particles of the third composition, and optionally the first and second compositions.

Starch Particles

The starch particles are comprised of starch, crosslinked starch, starch derivatives, or mixtures of these, and optionally these particles may be partially or fully coated to modify their surface properties. It is important for these starch particles to remain as discrete particles in the third composition of the present invention, and hence these starches are generally not subject to high temperatures for any extended period of time. The starch particles in the third composition have an average diameter of from about 5 μm to about 30 μm, preferably from about 8 μm to about 25 μm, from about 10 μm to about 20 μm.

Examples of commercially available starch particles include tapioca starch (available as Tapioca Pure from AkzoNobel), corn starch (available as Purity 21C from AkzoNobel), potato starch, glyceryl starch (available as Dry-Flo GS from AkzoNobel), aluminum starch octenylsuccinate (available as Mackaderm ASTO-Dry from Rhodia, Inc., and Dry-Flo PC from AkzoNobel), calcium starch octenylsuccinate (available as Skin Flow C from MGP Ingredients, Inc., and Mackaderm CSTO-Dry from Rhodia, Inc.), and polymethylsilsesquioxane coated tapioca starch (available as Dry-Flo TS from AkzoNobel).

### Spherical Silicone Elastomer Particles

The particles of the present invention may comprise spherical silicone elastomer particles that may optionally have polyorganosilsesquioxane attached on the surface. The silicone elastomer constituting the substantially spherical silicone elastomer particles preferably have no tackiness and the rubber hardness measured by Durometer A defined in JIS K 6253 is preferably in the range of 10 to 90, more preferably 20 to 80, and even more preferably 25 to 75. When the rubber hardness is less than 5, the resulting silicone particles tend to become agglomerated, and dispersion into primary particles will be difficult leading to a loss in feel and/or look benefits. The rubber hardness in excess of 90 will invite loss of soft texture. Silicone elastomer particles suitable for the present invention include silicone resin-coated silicone rubber particles. The spherical silicone elastomer particles in the third composition have an average diameter of from about 1 μm to about 40 μm, preferably from about 2 μm to about 30 μm, from about 5 μm to about 15 μm.

It is known in the prior art that silicone particles can be prepared from several silicone materials, e.g. organopolysiloxanes, including cured silicone rubbers. Commercially available silicone particles suitable for use in the present invention include: KSP-100, -101, -102, -103, -104, and -105, all from Shin Etsu; and, DC9506, and DC 9701 from Dow Corning.

### Optional Ingredients

Emulsifier

Any or a combination of the three compositions may comprise an emulsifier. An emulsifier is particularly suitable when the composition is in the form of an emulsion or if immiscible materials are being combined. The compositions may comprise from about 0.05%, 0.1%, 0.2%, 0.3%, 0.5%, or 1% to about 5%, 3%, 2%, or 1% emulsifier. Emulsifiers may be nonionic, anionic or cationic. Non-limiting examples of emulsifiers are disclosed in U.S. Pat. No. 3,755,560, U.S. Pat. No. 4,421,769, and McCutcheon's, Emulsifiers and Detergents, 2010 Annual Ed., published by M. C. Publishing Co. Other suitable emulsifiers are further described in the Personal Care Product Council's International Cosmetic Ingredient Dictionary and Handbook, Thirteenth Edition, 2006, under the functional category of “Surfactants-Emulsifying Agents.”

Suitable emulsifiers include the following classes of ethers and esters: ethers of polyglycols and of fatty alcohols; esters of polyglycols and of fatty acids; ethers of polyglycols and of fatty alcohols which are glycosylated; esters of polyglycols and of fatty acids which are glycosylated; ethers of C₁₂₋₃₀ alcohols and of glycerol or of polyglycerol; esters of C₁₂₋₃₀ fatty acids and of glycerol or of polyglycerol; ethers of oxyalkylene-modified C₁₂₋₃₀ alcohols and of glycerol or polyglycerol; ethers of C₁₂₋₃₀ fatty alcohols comprising and of sucrose or of glucose; esters of sucrose and of C₁₂₋₃₀ fatty acids; esters of pentaerythritol and of C₁₂₋₃₀ fatty acids; esters of sorbitol and/or of sorbitan and of C₁₂₋₃₀ fatty acids; ethers of sorbitol and/or of sorbitan and of alkoxylated sorbitan; ethers of polyglycols and of cholesterol; esters of C₁₂₋₃₀ fatty acids and of alkoxylated ethers of sorbitol and/or sorbitan; and combinations thereof.

Silicone emulsifiers may be use in the present compositions. Linear or branched type silicone emulsifiers may also be used. Particularly useful silicone emulsifiers include polyether modified silicones such as KF-6011, KF-6012, KF-6013, KF-6015, KF-6015, KF-6017, KF-6043, KF-6028, and KF-6038 and polyglycerolated linear or branched siloxane emulsifiers such as KF-6100, KF-6104, and KF-6105; all from Shin Etsu.

Volatile Oils

Any or a combination of the compositions comprise from about 0% to about 30%, or, alternatively, from about 5% to about 20% of one or more volatile oils. Suitable volatile oils include volatile silicones and volatile hydrocarbon oils.

Suitable volatile silicones include cyclic and linear volatile silicones. A description of various volatile silicones is found in Todd, et al. “Volatile Silicone Fluids for Cosmetics”, 91 Cosmetics and Toiletries 27-32 (1976). Suitable cyclic volatile silicones include cyclic dimethyl siloxane chains containing an average of from about 3 to about 5 silicon atoms, preferably from about 4 to about 5 silicon atoms. Exemplary cyclic volatile silicones of varying viscosities include Dow Corning DC 244, DC 245, DC 344, and DC 345; GE Silicones-OSi Specialties Volatile Silicone 7207 and Volatile Silicone 7158; and GE Silicones SF1202. Suitable volatile linear silicones include the polydimethylsiloxanes containing an average of from about 2 to about 8 silicon atoms. Exemplary linear volatile silicones include the Dow Corning DC 200 series with viscosities of 0.65 cst, 1.0 cst, and 2.0 cst. In certain embodiments, the linear volatile silicones generally have viscosities of less than or equal to about 4 centistokes at 25° C., and the cyclic materials generally have viscosities of less than about 6 centistokes at 25° C.

Also suitable volatile oils are volatile hydrocarbons. Examples of suitable volatile hydrocarbon oils include, but are not limited to, isododecane (e.g., Permethyl-99A which is available from Presperse Inc.), isodecane, and the C7-C8 through C12-C15 isoparaffins (e.g., Isopar Series available from Exxon Chemicals).

Thickening Agent

Any or a combination the three compositions may include one or more thickening agents. The compositions may comprise from about 0.1% to about 5%, or, alternatively, from about 0.3% to about 3%, of a thickening agent when present. Suitable classes of thickening agents include but are not limited to carboxylic acid polymers, polyacrylamide polymers, sulfonated polymers, copolymers thereof, hydrophobically modified derivatives thereof, and mixtures thereof.

Suitable thickening agents include carboxylic acid polymers such as the carbomers (e.g., the CARBOPOL® 900 series such as CARBOPOL® 954), and Ultrez 10 and Ultrez 30. Other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as CARBOPOL® 1342, CARBOPOL® 1382, Ultrez 20, Ultrez 21, PEMULEN TR-1, and PEMULEN TR-2, from Noveon, Inc.

Other suitable thickening agents include the polyacrylamide polymers and copolymers. An exemplary polyacrylamide polymer has the CTFA designation “polyacrylamide and isoparaffin and laureth-7” and is available under the trade name SEPIGEL 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500 W, SSSA100H, from Lipo Chemicals, Inc., (Patterson, N.J.).

Other suitable thickening agents useful herein are sulfonated polymers such as the CTFA designated sodium polyacryloyldimethyl taurate available under the trade name Simulgel 800 from Seppic Corp. and Viscolam At 100 P available from Lamberti S.p.A. (Gallarate, Italy). Another commercially available material comprising a sulfonated polymer is Sepiplus 400 available from Seppic Corp.

Further, suitable thickeners may include superabsorbent polymers. These superabsorbent polymers may be chosen from: crosslinked sodium polyacrylates, such as, for example, those sold under the names Octacare X100, X110 and RM100 by Avecia, those sold under the names Flocare GB300 and Flosorb 500 by SNF, those sold under the names Luquasorb 1003, Luquasorb 1010, Luquasorb 1280 and Luquasorb 1100 by BASF, those sold under the names Water Lock G400 and G430 (INCI name: Acrylamide/Sodium Acrylate Copolymer) by Grain Processing, or Aqua Keep 10 SH NF, Aqua Keep 10 SH NFC, sodium acrylate crosspolymer-2, provided by Sumitomo Seika, starches grafted by an acrylic polymer (homopolymer or copolymer) and in particular by sodium polyacrylate, such as those sold under the names Sanfresh ST-100C, ST100MC and IM-300MC by Sanyo Chemical Industries, Makimousse 12 and Makimouse 25 supplied by Kobo Products Inc (INCI name: Sodium Polyacrylate Starch), hydrolysed starches grafted by an acrylic polymer (homopolymer or copolymer), in particular the acryloacrylamide/sodium acrylate copolymer, such as those sold under the names Water Lock A-240, A-180, B-204, D-223, A-100, C-200 and D-223 by Grain Processing (INCI name: Starch/Acrylamide/Sodium Acrylate Copolymer). Preferred superabsorbent polymers include Makimousse 12 and Makimousse 25.

Suitable thickeners for use herein include gums. “Gum” is a broadly defined term in the art. Gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, derivatives thereof and mixtures thereof.

Natural gums are polysaccharides of natural origin, capable of causing a large viscosity increase in solution, even at small concentrations. They can be used as thickening agents, gelling agents, emulsifying agents, and stabilizers. Most often these gums are found in the woody elements of plants or in seed coatings. Natural gums can be classified according to their origin. They can also be classified as uncharged or ionic polymers (polyelectrolytes), examples of which include the following. Natural gums obtained from seaweeds, such as: agar; alginic acid; sodium alginate; and carrageenan. Natural gums obtained from non-marine botanical resources include: gum arabic, from the sap of Acacia trees; gum ghatti, from the sap of Anogeissus trees; gum tragacanth, from the sap of Astragalus shrubs; karaya gum, from the sap of Sterculia trees. Examples of uncharged gums include: guar gum, from guar beans, locust bean gum, from the seeds of the carob tree; beta-glucan, from oat or barley bran; chicle gum, an older base for chewing gum obtained from the chicle tree; dammar gum, from the sap of Dipterocarpaceae trees; glucomannan from the konjac plant; mastic gum, a chewing gum from ancient Greece obtained from the mastic tree; psyllium seed husks, from the Plantago plant; spruce gum, a chewing gum of American Indians obtained from spruce trees; tara gum, from the seeds of the tara tree. Natural gums produced by bacterial fermentation include gellan gum and xanthan gum.

Non-Spherical Silicone Elastomer

Any or a combination of the three compositions may comprise a non-spherical silicone elastomer. Silicone elastomers are useful for reducing the tackiness of the composition and for providing a pleasant feel upon application. One non-limiting example of a suitable class of silicone elastomers is crosslinked organopolysiloxane (or siloxane) elastomers, which are generally described in U.S. patent application publication US2003/0049212A1.

The compositions of the present invention may comprise from about 0.1% to about 4% or, alternatively, from about 0.5% to about 2% of one or more silicone elastomers. The indicated percentages are understood to refer to amount of dry elastomer, as opposed to the total amount of elastomer and solvent, used for example for storage and shipping.

Exemplary non-spherical crosslinked siloxane elastomers include the CTFA (Cosmetic, Toiletry, and Fragrance Association International Cosmetic Ingredient Dictionary and Handbook, 11^{th} ed.) designated dimethicone/vinyl dimethicone crosspolymers supplied by a variety of suppliers including Dow Corning™, General Electric™, Shin Etsu™ (KSG 15 and 16), and Grant Industries. Other exemplary non-emulsifying crosslinked siloxane elastomer include the CTFA designated dimethicone crosspolymers including Dow Corning™; e.g. DC 9040 and DC 9045 which are supplied as a 12.5% elastomers in cyclodimeticone, and DC 9041 which is supplied as 16% elastomer in dimeticone), as well as silicone organic elastomer blends including Dow Corning™; e.g. EL-8050 ID, EL-8051 IN, EL-8052 IH which are supplied as 14.25% elastomer in Isododecane, 12% elastomer in Isodecyl Neopentanoate, and 16% elastomer in isohexadecne, as well as hydro elastomer blends including Dow Corning™, e.g. EL-7040 which is supplied as 17.5% elastomer in caprylyl methicone.

UV Agents

The current compositions may contain a UV agent to provide UV protection. When present, the UV agent(s) present in the composition(s) herein may be added to provide a desired sun protection factor. For example, a composition herein may have a sun protection factor of 5 or more (e.g., 10, 15, 20, 25, 30, 35, 40, 50, 60, 70 80 90 or even up to 100) when used as intended on skin. The SPF of the composition may range from 4 to 100, from 8 to 55, or even from 12 to 35, as desired.

Some nonlimiting examples of known UV agents include Benzophenone, Benzophenone-1, Benzophenone-2, Benzophenone-3 (oxybenzone), Benzophenone-4 (sulisobenzone), Benzophenone-5, Benzophenone-6, Benzophenone-7, Benzophenone-8, Benzophenone-9 (dioxybenzone), Benzophenone-10, Benzophenone-11, Benzophenone-12, Benzotriazolyl Dodecyl p-Cresol, 3-Benzylidene Camphor, Benzylidene Camphor Sulfonic Acid, Benzyl Salicylate, Bis-Ethylhexyloxyphenol Methoxyphenyl Triazine (bemotrizinol), Bomelone, Bumetrizole, Butyl Methoxydibenzoylmethane (avobenzone), Butyl PABA, Cinnamidopropyltrimonium Chloride, Cinoxate, DEA-Methoxycinnamate, Dibenzoxazoyl Naphthalene, Di-t-Butyl Hydroxybenzylidene Camphor, Diethylamino Hydroxybenzoyl Hexyl Benzoate, Diethylhexyl Butamido Triazone (iscotrizinol), Diisopropyl Ethyl Cinnamate, Diisopropyl Methyl Cinnamate, Di-Methoxycinnamidopropyl Ethyldimonium Chloride Ether, Dimethyl PABA Ethyl Cetearyldimonium Tosylate, Dimorpholinopyridazinone, Dimorpholino-pryridazinone, Disodium Bisethylphenyl Triaminotriazine Stilbenedisulfonate, Disodium Distyrylbiphenyl Disulfonate, Disodium Phenyl Dibenzimidazole Tetrasulfonate, Drometrizole, Drometrizole Trisiloxane, Ethyl Dihydroxypropyl PAB A, Ethyl Diisopropylcinnamate, Ethylhexyl Bis-Isopentylbenzoxazolylphenyl Melamine, Ethyl Dimethoxybenzylidene Dioxoimidazolidine Propionate, Ethylhexyl Dimethyl PABA, Ethylhexyl Methoxycinnamate (octinoxate), Ethylhexyl Methoxydibenzoylmethane, Ethylhexyl Salicylate (octisalate), Ethylhexyl Triazone (octyl triazone), Ethyl Methoxycinnamate, Ethyl PABA, Ethyl Urocanate, Etocrylene, 4-(2-Beta-Glucopyrano-siloxy) Propoxy-2-Hydroxybenzophenone, Glyceryl Ethylhexanoate Dimethoxycinnamate, Glyceryl PABA, Glycol Salicylate, Hexanediol Disalicylate, Homosalate, Isoamyl Cinnamate, Isoamyl p-Methoxycinnamate, Isopentyl Trimethoxycinnamate Trisiloxane, Isopropylbenzyl Salicylate, Isopropyl Dibenzoylmethane, Isopropyl Methoxy-cinnamate, Kaempferia Galanga Root Extract, Menthyl Anthranilate (meradimate), Menthyl Salicylate, Methoxycinnamidopropyl Hydroxysultaine, Methoxycinnamidopropyl Laurdimonium Tosylate, 4-Methylbenzylidene Camphor (enacamene), Methylene Bis-Benzotriazolyl Tetramethylbutylphenol (bisoctrizole), Octocrylene, Octrizole, PABA, PEG-25 PABA, Phenylbenzimidazole Sulfonic Acid (ensulizole), Polyacrylamidomethyl Benzylidene Camphor, Polyamide-2, Polyquatemium-59, Polysilicone-15 (diethylbenzylidene malonate dimethicone), Potassium Methoxy-cinnamate, Potassium Phenylbenzimidazole Sulfonate, Red Petrolatum, Sodium Benzotriazoyl Butylphenol Sulfonate, Sodium Phenylbenzimidazole Sulfonate, Sodium Urocanate, TEA-Phenylbenzimidazole Sulfonate, TEA-Salicylate, Terephthalylidene Dicamphor Sulfonic Acid (ecamsule), Tetrabutyl Phenyl Hydroxybenzoate, Titanium Dioxide, Urocanic Acid, Zinc Cerium Oxide, Zinc Oxide

Some particularly suitable examples of UV agents that are generally recognized as being liquids are Ethylhexyl Dimethyl PABA, Ethylhexyl Methoxycinnamate (octinoxate), Ethylhexyl Salicylate (octisalate), Homosalate, Isoamyl p-Methoxycinnamate (amiloxate), Menthyl Anthranilate (meradimate), Octocrylene, Polysilicone-15 (diethylbenzylidene malonate dimethicone) and combinations of these.

Some particularly suitable examples of oil soluble solid UV agents, which can be dissolved in a non-volatile oil such as one of the UV agents described above to form a liquid UV agent, are Benzophenone-3 (oxybenzone), Benzophenone-9 (dioxybenzone), Bis-Ethylhexyloxyphenol Methoxyphenyl Triazine (bemotrizinol), Butyl Methoxydibenzoylmethane (avobenzone), Diethylamino Hydroxybenzoyl Hexyl Benzoate, Diethylhexyl Butamido Triazone (iscotrizinol), Drometrizole Trisiloxane, Ethylhexyl Triazone (octyl triazone), 4-Methylbenzylidene Camphor (enacamene) and combinations of these.

Actives

Any or a combination of the three compositions may comprise at least one skin care active (“active”), useful for regulating and/or improving the condition of mammalian skin. The active may be soluble in oil or water, and may be present primarily in the oil phase and/or in the aqueous phase. Solubility in water and oil is within the knowledge of one of skill in the art, and can be determined using known methods of analysis. One of skill in the art further will understand that solubility may be affected by the type and concentration of other components in the composition, and other conditions such as pH, ionic strength, etc. Many skin care actives may provide more than one benefit, or operate via more than one mode of action; therefore, classifications herein are made for the sake of convenience and are not intended to limit the active to that particular application or applications listed. Suitable actives include, but are not limited to, vitamins, peptides, sugar amines, oil control agents, tanning actives, anti-acne actives, desquamation actives, anti-cellulite actives, chelating agents, skin lightening agents, flavonoids, protease inhibitors, non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobials, and antifungals.

Any or a combination of the three compositions may comprise from about 0.001% to about 10%, alternatively from about 0.01% to about 5%, of at least one vitamin. Herein, “vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C1-C18 nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

Any or a combination of the three compositions may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, carnosine (beta-alanine-histidine), palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, acetyl-glutamate-glutamate-methionine-glutamine-arginine-arginine (Ac-EEMQRR; Argireline®), and Cu-histidine-glycine-glycine (Cu-HGG, also known as IAMIN®). The compositions may comprise from about 1×10⁻⁷% to about 20%, alternatively from about 1×10⁻⁶% to about 10%, and alternatively from about 1×10⁻⁵% to about 5% of the peptide.

### Test Methods

Chroma Method

Herein, “chroma” describes color and color intensity. For the purposes of the present invention, color is defined according to a value on the CIELAB color system, which is based on the XYZ color system, defined by the Commission Internationale de l'Eclairage (CIE system) to provide a manner of objectively representing perceived color and color differences. X, Y and Z can be expressed in a variety of manners, or “scales,” one of which is the Hunter scale. The Hunter scale has three variables, L, a, and b, which correlate mathematically to X, Y and Z, and is described by Robertson, A. R. in “The CIE 1976 Color Difference Formulas,” Color Research Applications, vol. 2, pp. 7-11 (1977).

To measure the color of the compositions of the present invention, a thick, uniform film of the composition is first created on a standard background. Specifically, product is applied onto a standard opacity chart (Form N2A, Leneta Company of Manwah, N.J. or the equivalent thereof, of which the top half is black and the bottom half is white) and then spread on the black area of the opacity chart into a film having a thickness of approximately 0.01 inches using a film applicator (e.g., as commercially available from BYK Gardner of Columbia, Md., or the equivalent thereof).

The color (L, a, and b values) of the product film is then measured using a spectrophotometer with settings selected to exclude specular reflection. The value for “a” correlates to a value along the red-green (horizontal) axis, and the value for “b” correlates to a value along the blue-yellow (vertical) axis. For example, a blue-colored sample will have a negative b-value, whereas a red-colored sample will have a positive a-value. A more positive or negative value represents a more intense color. The value for “L” is an indicator of lightness and/or darkness, and correlates to a value along the z-axis, which is perpendicular to both the horizontal and vertical axes.

“Chroma” is measured by a vector having its origin at the intersection of the red-green and blue-yellow axes and extending outward into the color space defined by the horizontal and vertical axes of the CIELAB color system. The length of the vector represents the chroma, and the direction of the vector represents the shade, or hue. The shorter the vector, the less colored is the composition, and the lower the chroma. The chroma for the individual layers of the present invention, that is the chroma value for the first layer or the third layer is less than about 10, preferably less than about 6, and even more preferably less than about 3.

Contrast Ratio

Herein, “contrast ratio” refers to the opacity of the composition, or the ability of the composition to reduce or prevent light transmission, determined after the composition is drawn onto an opacity chart (Form N2A, Leneta Company of Manwah, N.J. or the equivalent thereof), and by using a spectrophotometer with settings selected to exclude specular reflection. The composition is applied to the top of the opacity chart and then is drawn into a film having a thickness of approximately 0.0015 inches using a film applicator (e.g., as commercially available from BYK Gardner of Columbia, Md., or the equivalent thereof). The film is allowed to dry for 2 hours under conditions of 22° C.+/−1° C., 1 atm. Using a spectrophotometer, the Y tristimulus value (i.e., the XYZ color space of the film) of the product film is measured and recorded. The Y tristimulus value is measured in three different areas of the product film over the black section of the opacity chart, and also in three different areas of the product film over the white section of the opacity chart.

The contrast ratio for the individual layers of the present invention, that is the contrast ratio for the first and second compositions is less than about 20, preferably less than about 10, and even more preferably less than about 6.

The contrast ratio is calculated as the mathematical average of the three Y tristimulus values over the black areas, divided by the mathematical average of the three Y tristimulus values over the white areas, times 100:

$$Contrast Ratio = \frac{average ( Y black )}{average ( Y white )} \times 100$$

Visual Attribute Test (VAT)

The visible attribute test (VAT) is a technical panel used to quantify visible benefits of the compositions of the present invention when applied to facial skin. Fifteen to thirty female panelists who are pre-screened to have moderate or higher baseline levels of facial attributes such as fine lines, wrinkles, bumpy surface texture, and pores participate in each VAT study. Two trained expert graders then grade various attributes on each panelist's face both at baseline and 10 minutes after application of 0.45 grams of product to one side of the face. Reductions in facial attributes are then calculated as pre-treatment grade minus the post-treatment grade, and the significance of the differences are determined using ANOVA procedures (Tukey's LSD test).

A hypothetical data table representing typical VAT data calculations for bumpy surface texture is below. For fine lines, wrinkles, bumpy surface texture and/or pores, a difference of greater than approximately 0.4 provides consumer noticeable changes.

| Panelist | Pre-Treatment | Post-Treatment | Delta |
|---|---|---|---|
| Number | Grade | Grade | (Pre Minus Post) |
| 1 | 3.65 | 3.15 | 0.5 |
| 2 | 3.5 | 2.95 | 0.55 |
| 3 | 4.1 | 3.2 | 0.9 |
| 4 | 4.5 | 3.85 | 0.65 |
| 5 | 3.7 | 2.8 | 0.9 |

The facial attributes evaluated by the expert graders include the following:

Lines/Wrinkles—Severity of the skin on the cheek areas caused by fine lines and wrinkles. The cheek area includes that which is below the top of the cheek bone, excluding skin around the mouth. Features of this attribute include the number, length, depth, and percent coverage of the lines and wrinkles. (Does NOT encompass pores directly, but does include lines which appear to be formed by interconnected pores.) Each of the features of this attribute are equally weighted.

Bumpy Surface—Skin unevenness or roughness associated with a “pebbled” or an “orange peel” surface. Based on both the degree of roughness as defined as height and proximity and the percentage of the face covered by the surfaced appearance. Roughness and coverage are equally weighted in the final grade. (Does NOT include obviously raised brown moles.)

Pores—Coverage and intensity of the facial pores. Coverage is defined as the percentage of the entire cheek areas that possess visible pores (open holes). Intensity is defined as the quantity of pores and the average pore size where larger pores drive higher scores. Both elements of this attribute are equally weighted in the final grade.

Brightness—The brightening feature increases the lightness and luminescence of the face. It may be accompanied by a reduction in red and brown tones.

The expert graders rate each of the above attributes both pre- and post-treatment using the 5 point continuous line scale shown below:

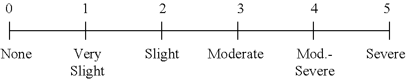

### EXAMPLES

### Examples 1-10

### First Compositions

The following examples of the first compositions for forming the first layer according to the present invention are prepared by first combining the water phase ingredients and mixing until uniform, warming if necessary. Next, the thickeners are added and the composition is again mixed until uniform. Finally, the pH adjustor, if present, is added and the composition is mixed until uniform.

| | Exam- | Exam- | Exam- | Exam- | Exam- |
|---|---|---|---|---|---|
| | ple 1 | ple 2 | ple 3 | ple 4 | ple 5 |
| | First | First | First | First | First |
| | Comp. | Comp. | Comp. | Comp. | Comp. |
| Water Phase: | | | | | |
| Water | Qs | qs | qs | qs | Qs |
| Glycerin | 25.0 | 15.0 | 25.0 | 15.0 | 25.0 |
| Dipropylene Glycol | — | — | — | — | 5.0 |
| Butylene Glycol | — | — | — | 3.0 | — |
| Disodium EDTA | 0.05 | 0.025 | 0.025 | 0.05 | 0.05 |
| Symdiol 68¹ | — | 0.7 | — | 0.7 | 0.7 |
| Glycacil L² | — | 0.09 | — | 0.09 | 0.09 |
| Phenoxyethanol | — | — | — | 0.5 | — |
| Glydant Plus | 0.3 | — | 0.3 | — | — |
| Liquid³ | | | | | |
| Niacinamide | 5.0 | 5.0 | 5.0 | 3.5 | 1.0 |
| D-panthenol | 1.0 | 0.5 | 1.0 | 0.25 | — |
| Sepiwhite MSH⁴ | 1.0 | — | — | — | — |
| Glyco-Repair⁵ | — | — | — | 1.0 | 2.0 |
| Biomyox⁶ | 2.0 | — | — | — | 1.0 |
| Palestrina⁷ | 3.35 | — | — | — | — |
| N-acetyl | — | — | — | 2.0 | 0.5 |
| glucosamine | | | | | |
| Inositol | — | — | — | 0.5 | — |
| Olivem 460⁸ | 0.1 | — | — | 0.1 | — |
| Aloe Vera Gel | — | — | — | — | 0.5 |
| Green Tea Extract | — | — | — | 1.0 | — |
| pH Adjustor: | | | | | |
| Triethanolamine | 0.45 | — | — | — | 0.2 |
| Thickener: | | | | | |
| Sepigel 305⁹ | — | — | — | 2.4 | — |
| Simulgel | — | 2.0 | 2.0 | — | 1.0 |
| INS-100¹⁰ | | | | | |
| Makimousse-12¹¹ | 1.0 | — | — | — | — |
| Ultrez 10¹² | — | — | — | — | 0.2 |
| Total: | 100% | 100% | 100% | 100% | 100% |

¹1,2-hexanediol and caprylyl glycol, from Symrise
²Iodopropynyl butylcarbamate, PEG-4 laurate, PEG-4 dilaurate, and polyethylene glycol, from Lonza
³DMDM Hydantoin, Butane-1,3-diol, iodopropynyl butylcarbamate, water, from Lonza
⁴Undecylenoyl phenylalanine, from Seppic
⁵Water and hydrolyzed ceratonia siliqua seed extract, from Silab
⁶Water and nasturtrium officinale extract, from Silab
⁷Water, glycerin, decyl glucoside, lactic acid, benzyl alcohol, and palmitoyl dipeptide-7, from Sederma (France)
⁸Sodium PEG-7 olive oil carboxylate, from B&T S.r.l.
⁹Polyacrylamide, C13-14 isoparaffin, and laureth-7, from Seppic
¹⁰Sodium acrylate/sodium acryloyldimethyl taurate copolymer, isohexadecane, and polysorbate 60, from Seppic
¹¹Sodium polyacrylate starch, from Kobo Products Inc.
¹²Carbomer, from Lubrizol

| | Exam- | Exam- | Exam- | Exam- | Exam- |
|---|---|---|---|---|---|
| | ple 6 | ple 7 | ple 8 | ple 9 | ple 10 |
| | First | First | First | First | First |
| | Comp. | Comp. | Comp | Comp. | Comp. |
| Water Phase: | | | | | |
| Water | Qs | qs | qs | qs | Qs |
| Glycerin | 15.0 | 15.0 | 10.0 | 10.0 | 10.0 |
| Propylene Glycol | 3.0 | 5.0 | — | 5.0 | 5.0 |
| Dipropylene Glycol | 3.0 | — | 10.0 | 5.0 | 2.0 |
| Butylene Glycol | 3.0 | — | — | 5.0 | — |
| Disodium EDTA | 0.05 | 0.05 | 0.05 | 0.05 | 0.05 |
| Methylparaben | 0.1 | — | — | — | — |
| Symdiol 68¹ | — | 0.7 | 0.7 | 0.7 | — |
| Sodium Benzoate | 0.1 | — | — | — | — |
| Glycacil L² | — | 0.09 | 0.09 | 0.09 | — |
| Phenoxyethanol | 0.5 | 0.1 | — | — | — |
| Glydant Plus | — | — | — | — | 0.3 |
| Liquid³ | | | | | |
| Niacinamide | 3.0 | 2.0 | — | — | — |
| D-panthenol | 0.7 | 0.3 | 0.5 | — | — |
| Glyco-Repair⁴ | — | — | — | — | 1.0 |
| Biomyox⁵ | — | 1.5 | — | — | — |
| Palmitoyl- | 0.03 | — | — | — | 0.01 |
| pentapeptide⁶ | | | | | |
| N-acetyl | — | — | 1.0 | — | — |
| glucosamine | | | | | |
| Aloe Vera Gel | — | — | — | — | 0.1 |
| Green Tea Extract | — | 0.5 | — | — | 0.1 |
| pH Adjustor: | | | | | |
| Triethanolamine | — | 0.3 | — | — | — |
| Aminomethyl | — | — | — | 0.1 | 0.1 |
| propanol | | | | | |
| Thickener: | | | | | |
| Simulgel | 2.5 | — | — | 0.5 | — |
| INS-100⁷ | | | | | |
| Makimousse-7⁸ | — | — | — | — | 0.6 |
| Makimousse-12⁹ | — | — | 0.4 | 0.3 | — |
| Ultrez 10¹⁰ | — | — | — | 0.1 | — |
| Ultrez 21¹¹ | — | 0.3 | — | — | — |
| Xanthan gum | — | — | 0.1 | — | — |
| Total: | 100% | 100% | 100% | 100% | 100% |

¹1,2-hexanediol and caprylyl glycol, from Symrise
²Iodopropynyl butylcarbamate, PEG-4 laurate, PEG-4 dilaurate, and polyethylene glycol, from Lonza
³DMDM Hydantoin, Butane-1,3-diol, iodopropynyl butylcarbamate, water, from Lonza
⁴Water and hydrolyzed ceratonia siliqua seed extract, from Silab
⁵Water and nasturtrium officinale extract, from Silab
⁶Palmitoyl-lysine-threonine-threonine-lysine-serine available from Sederma (France)
⁷Sodium acrylate/sodium acryloyldimethyl taurate copolymer, isohexadecane, and polysorbate 60, from Seppic
⁸Sodium polyacrylate starch, from Kobo Products Inc.
⁹Sodium polyacrylate starch, from Kobo Products Inc.
¹⁰Carbomer, from Lubrizol
¹¹Acrylates C10-/30 alkyl acrylate crosspolymer, from Lubrizol

### Examples 11-20

### Second Compositions

The following examples of the second compositions for forming the second layer according to the present invention are prepared by first combining the water phase ingredients in a container and mixing until uniform. The thickener is added and the water phase is mixed until uniform, and then the pH adjuster, if present, is added and the water phase is again mixed until uniform. The oil phase ingredients are combined in a separate container and mixed until uniform. Finally, the oil phase is added to the water phase and the resulting emulsion is subjected to high shear mixing (e.g., Flacktek Speedmixer, or rotor-stator mill)

| | Exam- | Exam- | Exam- | Exam- | Exam- |
|---|---|---|---|---|---|
| | ple 11 | ple 12 | ple 13 | ple 14 | ple 15 |
| | Second | Second | Second | Second | Second |
| | Comp. | Comp. | Comp. | Comp. | Comp. |
| Water Phase: | | | | | |
| Water | Qs | qs | qs | qs | qs |
| Disodium EDTA | 0.05 | 0.05 | 0.05 | 0.05 | 0.05 |
| Methylparaben | — | — | — | 0.1 | — |
| Symdiol 68¹ | — | 0.7 | — | — | — |
| Glycacil L² | — | 0.09 | — | 0.09 | — |
| Phenoxyethanol | — | — | — | 0.5 | — |
| Glydant Plus | 0.3 | — | 0.3 | — | 0.3 |
| Liquid³ | | | | | |
| pH Adjustor: | | | | | |
| Triethanolamine | — | — | — | 0.2 | — |
| Thickener: | | | | | |
| Sepigel 305⁴ | — | — | — | 1.0 | — |
| Simulgel | 2.0 | 2.0 | 2.0 | — | — |
| INS-100⁵ | | | | | |
| Makimousse-12⁶ | — | — | — | — | 0.4 |
| Ultrez 10⁷ | — | — | — | 0.1 | — |
| Ultrez 21⁸ | — | — | — | 0.1 | — |
| Xanthan gum | — | — | — | — | 0.1 |
| Oil Phase: | | | | | |
| Cyclomethicone D5 | 24.2 | 8.0 | 24.2 | — | 14.0 |
| Dimethicone 2 cst | — | — | — | 15.0 | — |
| Dimethicone 20 cst | — | — | — | — | — |
| Dimethicone 50 cst | 4.39 | 10.0 | 1.89 | 10.0 | 8.0 |
| Dimethicone 350 cst | — | — | — | — | — |
| DE-23⁹ | — | — | — | — | — |
| DC 5562¹⁰ | — | — | — | — | 2.0 |
| DC1503¹¹ | — | — | — | — | 2.0 |
| DC9045¹² | 11.0 | 11.0 | 11.0 | — | — |
| Isopropyl lauroyl | 7.32 | — | 4.82 | — | — |
| sarcosinate | | | | | |
| Hexyldecanol | — | — | 5.0 | — | — |
| Laureth-4 | 0.2 | 0.2 | 0.2 | 0.3 | 0.3 |
| Total: | 100% | 100% | 100% | 100% | 100% |

¹1,2-hexanediol and caprylyl glycol, from Symrise
²Iodopropynyl butylcarbamate, PEG-4 laurate, PEG-4 dilaurate, and polyethylene glycol, from Lonza
³DMDM Hydantoin, Butane-1,3-diol, iodopropynyl butylcarbamate, water, from Lonza
⁴Polyacrylamide, C13-14 isoparaffin, and laureth-7, from Seppic
⁵Sodium acrylate/sodium acryloyldimethyl taurate copolymer, isohexadecane, and polysorbate 60, from Seppic
⁶Sodium polyacrylate starch, from Kobo Products Inc.
⁷Carbomer, from Lubrizol
⁸Acrylates C10-/30 alkyl acrylate crosspolymer, from Lubrizol
⁹Polydiethylsiloxane, from Gelest
¹⁰Bis-hydroxyethoxylpropyl dimethicone, from Dow Corning
¹¹Dimethicone and dimethiconol, from Dow Corning
¹²Cyclopentasiloxane and dimethicone crosspolymer, from Dow Corning

| | Exam- | Exam- | Exam- | Exam- | Exam- |
|---|---|---|---|---|---|
| | ple 16 | ple 17 | ple 18 | ple 19 | ple 20 |
| | Second | Second | Second | Second | Second |
| | Comp. | Comp. | Comp. | Comp. | Comp. |
| Water Phase: | | | | | |
| Water | Qs | qs | qs | qs | Qs |
| Disodium EDTA | 0.05 | 0.05 | 0.05 | 0.05 | 0.05 |
| Methylparaben | — | — | — | 0.1 | — |
| Symdiol 68¹ | 0.7 | 0.7 | 0.7 | — | — |
| Glycacil L² | 0.09 | 0.09 | 0.09 | 0.09 | — |
| Phenoxyethanol | — | — | 0.2 | 0.5 | — |
| Glydant Plus | — | — | — | — | 0.3 |
| Liquid³ | | | | | |
| pH Adjustor: | | | | | |
| Triethanolamine | — | — | — | 0.2 | — |
| Thickener: | | | | | |
| Sepigel 305⁴ | — | — | 0.6 | 1.0 | — |
| Simulgel | 2.0 | 1.5 | 1.0 | — | 0.7 |
| INS-100⁵ | | | | | |
| Makimousse-12⁶ | — | — | — | — | 0.3 |
| Ultrez 10⁷ | — | — | — | 0.1 | — |
| Ultrez 21⁸ | — | — | — | 0.1 | — |
| Xanthan gum | — | — | — | — | 0.1 |
| Oil Phase: | | | | | |
| Cyclomethicone D5 | 18.0 | 16.0 | — | 16.0 | 18.0 |
| Dimethicone 2 cst | — | — | 12.0 | — | — |
| Dimethicone 20 cst | — | — | 3.0 | — | — |
| Dimethicone 50 cst | 8.0 | 10.0 | — | 6.0 | 6.0 |
| Dimethicone 350 cst | — | — | 3.0 | 2.0 | — |
| DE-23⁹ | — | — | 3.0 | — | — |
| DC 5562¹⁰ | — | — | — | — | 2.0 |
| DC1503¹¹ | — | — | — | — | 2.0 |
| DC9045¹² | — | 4.0 | — | — | — |
| Laureth-4 | 0.3 | 0.3 | 0.3 | 0.3 | 0.3 |
| Total: | 100% | 100% | 100% | 100% | 100% |

¹1,2-hexanediol and caprylyl glycol, from Symrise
²Iodopropynyl butylcarbamate, PEG-4 laurate, PEG-4 dilaurate, and polyethylene glycol, from Lonza
³DMDM Hydantoin, Butane-1,3-diol, iodopropynyl butylcarbamate, water, from Lonza
⁴Polyacrylamide, C13-14 isoparaffin, and laureth-7, from Seppic
⁵Sodium acrylate/sodium acryloyldimethyl taurate copolymer, isohexadecane, and polysorbate 60, from Seppic
⁶Sodium polyacrylate starch, from Kobo Products Inc.
⁷Carbomer, from Lubrizol
⁸Acrylates C10-/30 alkyl acrylate crosspolymer, from Lubrizol
⁹Polydiethylsiloxane, from Gelest
¹⁰Bis-hydroxyethoxylpropyl dimethicone, from Dow Corning
¹¹Dimethicone and dimethiconol, from Dow Corning
¹²Cyclopentasiloxane and dimethicone crosspolymer, from Dow Corning

### Examples 21-30

### Third Compositions

The following examples of the third compositions for forming the third layer according to the present invention are prepared by first combining the water phase ingredients in a container and mixing until uniform. The thickener is added and the water phase is mixed until uniform, and the pH adjustor, if present, is added and the water phase is again mixed until uniform. The oil phase ingredients, if present, are combined in a separate container and mixed until uniform. The oil phase, if present, is then added to the water phase and mixed until uniform. The powders are then added to the water phase and mixed until uniform. Finally, the resulting product is subjected to high shear mixing (e.g., Flacktek Speedmixer, or rotor-stator mill)

| | Exam- | Exam- | Exam- | Exam- | Exam- |
|---|---|---|---|---|---|
| | ple 21 | ple 22 | ple 23 | ple 24 | ple 25 |
| | Third | Third | Third | Third | Third |
| | Comp. | Comp. | Comp. | Comp. | Comp. |
| Water Phase: | | | | | |
| Water | Qs | qs | qs | qs | qs |
| Disodium EDTA | 0.05 | 0.05 | 0.05 | 0.05 | 0.05 |
| Methylparaben | — | — | — | 0.1 | — |
| Symdiol 68¹ | — | 0.7 | — | — | — |
| Glycacil L² | — | 0.09 | — | 0.09 | — |
| Phenoxyethanol | — | — | — | 0.5 | — |
| Glydant Plus | 0.3 | — | 0.3 | — | 0.3 |
| Liquid³ | | | | | |
| pH Adjustor: | | | | | |
| Triethanolamine | — | — | — | 0.2 | — |
| Thickener: | | | | | |
| Sepigel 305⁴ | — | — | — | 1.0 | — |
| Simulgel | 2.0 | 2.0 | 2.0 | — | — |
| INS-100⁵ | | | | | |
| Makimousse-12⁶ | — | — | — | — | 0.4 |
| Ultrez 10⁷ | — | — | — | 0.1 | — |
| Ultrez 21⁸ | — | — | — | 0.1 | — |
| Xanthan gum | — | — | — | — | 0.1 |
| Oil Phase: | | | | | |
| Cyclomethicone D5 | — | — | 6.0 | 15.0 | 14.0 |
| Dimethicone 2 cst | — | — | — | — | — |
| Laureth-4 | — | — | — | 0.3 | 0.3 |
| Particles: | | | | | |
| Dry Flo TS⁹ | — | 20.0 | — | — | — |
| Tapioca Pure¹⁰ | — | — | — | 10.0 | — |
| Dry Flo Pure¹¹ | — | — | — | — | 5.0 |
| KSP 100¹² | — | — | — | — | 10.0 |
| KSP 101¹³ | — | — | — | 5.0 | — |
| KSP 102¹⁴ | 11.0 | — | 11.0 | — | — |
| KSP 103¹⁵ | — | — | — | 5.0 | — |
| KSP 105¹⁶ | 11.0 | — | 11.0 | — | 5.0 |
| DC9506¹⁷ | — | — | — | — | — |
| Total: | 100% | 100% | 100% | 100% | 100% |

¹1,2-hexanediol and caprylyl glycol, from Symrise
²Iodopropynyl butylcarbamate, PEG-4 laurate, PEG-4 dilaurate, and polyethylene glycol, from Lonza
³DMDM Hydantoin, Butane-1,3-diol, iodopropynyl butylcarbamate, water, from Lonza
⁴Polyacrylamide, C13-14 isoparaffin, and laureth-7, from Seppic
⁵Sodium acrylate/sodium acryloyldimethyl taurate copolymer, isohexadecane, and polysorbate 60, from Seppic
⁶Sodium polyacrylate starch, from Kobo Products Inc.
⁷Carbomer, from Lubrizol
⁸Acrylates C10-/30 alkyl acrylate crosspolymer, from Lubrizol
⁹Tapioca and polymethylsilsesquioxane, from Akzo Nobel
¹⁰Tapioca powder, from Akzo Nobel
¹¹Aluminum starch octenyl succinate, from Akzo Nobel
¹²Vinyl dimethicone/methicone silsesquioxane crosspolymer, from Shin Etsu
¹³Vinyl dimethicone/methicone silsesquioxane crosspolymer, from Shin Etsu
¹⁴Vinyl dimethicone/methicone silsesquioxane crosspolymer, from Shin Etsu
¹⁵Vinyl dimethicone/methicone silsesquioxane crosspolymer, from Shin Etsu
¹⁶Vinyl dimethicone/methicone silsesquioxane crosspolymer, from Shin Etsu
¹⁷Dimethicone/Vinyl dimethicone crosspolymer, from Dow Corning

| | Exam- | Exam- | Exam- | Exam- | Exam- |
|---|---|---|---|---|---|
| | ple 26 | ple 27 | ple 28 | ple 29 | ple 30 |
| | Third | Third | Third | Third | Third |
| | Comp. | Comp. | Comp. | Comp. | Comp. |
| Water Phase: | | | | | |
| Water | Qs | qs | qs | qs | Qs |
| Disodium EDTA | 0.05 | 0.05 | 0.05 | 0.05 | 0.05 |
| Methylparaben | — | — | — | 0.1 | — |
| Symdiol 68¹ | 0.7 | 0.7 | 0.7 | — | — |
| Glycacil L² | 0.09 | 0.09 | 0.09 | 0.09 | — |
| Phenoxyethanol | — | — | 0.2 | 0.5 | — |
| Glydant Plus | — | — | — | — | 0.3 |
| Liquid³ | | | | | |
| pH Adjustor: | | | | | |
| Triethanolamine | — | — | — | 0.2 | — |
| Thickener: | | | | | |
| Sepigel 305⁴ | — | — | 0.6 | 1.0 | — |
| Simulgel | 2.0 | 1.5 | 1.0 | — | 0.7 |
| INS-100⁵ | | | | | |
| Makimousse-12⁶ | — | — | — | — | 0.3 |
| Ultrez 10⁷ | — | — | — | 0.1 | — |
| Ultrez 21⁸ | — | — | — | 0.1 | — |
| Xanthan gum | — | — | — | — | 0.1 |
| Oil Phase: | | | | | |
| Cyclomethicone D5 | 18.0 | 16.0 | — | 16.0 | 18.0 |
| Dimethicone 2 cst | — | — | 12.0 | — | — |
| Laureth-4 | 0.3 | 0.3 | 0.3 | 0.3 | 0.3 |
| Particles: | | | | | |
| KSP 100⁹ | 15.0 | — | 10 | 5.0 | — |
| KSP 101¹⁰ | — | — | — | 5.0 | — |
| KSP 102¹¹ | — | 10.0 | — | — | — |
| KSP 103¹² | — | — | — | 5.0 | — |
| KSP 105¹³ | — | 10.0 | 10 | — | 6.0 |
| DC9506¹⁴ | — | — | — | — | 10.0 |
| Total: | 100% | 100% | 100% | 100% | 100% |

¹1,2-hexanediol and caprylyl glycol, from Symrise
²Iodopropynyl butylcarbamate, PEG-4 laurate, PEG-4 dilaurate, and polyethylene glycol, from Lonza
³DMDM Hydantoin, Butane-1,3-diol, iodopropynyl butylcarbamate, water, from Lonza
⁴Polyacrylamide, C13-14 isoparaffin, and laureth-7, from Seppic
⁵Sodium acrylate/sodium acryloyldimethyl taurate copolymer, isohexadecane, and polysorbate 60, from Seppic
⁶Sodium polyacrylate starch, from Kobo Products Inc.
⁷Carbomer, from Lubrizol
⁸Acrylates C10-/30 alkyl acrylate crosspolymer, from Lubrizol
⁹Vinyl dimethicone/methicone silsesquioxane crosspolymer, from Shin Etsu
¹⁰Vinyl dimethicone/methicone silsesquioxane crosspolymer, from Shin Etsu
¹¹Vinyl dimethicone/methicone silsesquioxane crosspolymer, from Shin Etsu
¹²Vinyl dimethicone/methicone silsesquioxane crosspolymer, from Shin Etsu
¹³Vinyl dimethicone/methicone silsesquioxane crosspolymer, from Shin Etsu
¹⁴Dimethicone/Vinyl dimethicone crosspolymer, from Dow Corning

### Examples A through C

### Comparative Examples of All-in-One Compositions

The following are comparative examples of all-in-one compositions to compare to the three layer compositions of this invention. These all-in-one compositions are prepared by first combining the water phase ingredients in a container and mixing until uniform. The thickener is added and the water phase is mixed until uniform. The oil phase ingredients are combined in a separate container and mixed until uniform. The powders are next added to the oil phase and the combination is mixed until uniform. Finally, the oil/powder phase is added to the water phase and the resulting emulsion is subjected to high shear mixing (e.g., Flacktek Speedmixer, or rotor-stator mill)

| | Example A | Example B | Example C |
|---|---|---|---|
| | All-in-One | All-in-One | All-in-One |
| | Composition | Composition | Composition |
| Water Phase: | | | |
| Water | Qs | Qs | Qs |
| Glycerin | 25.0 | 5.0 | 15.0 |
| Disodium EDTA | 0.05 | 0.05 | 0.05 |
| Symdiol 68¹ | — | — | 0.7 |
| Glycacil L² | — | — | 0.09 |
| Glydant Plus | 0.3 | 0.3 | — |
| Liquid³ | | | |
| Niacinamide | 2.0 | 2.0 | 5.0 |
| D-panthenol | 0.5 | 0.5 | 0.5 |
| Polysorbate 60 | 0.2 | 0.2 | — |
| Thickener: | | | |
| Simulgel | 2.0 | 2.0 | 1.6 |
| INS-100⁴ | | | |
| Oil Phase: | | | |
| Cyclomethicone D5 | 24.2 | 24.2 | 8.0 |
| Dimethicone 50 cst | 4.39 | 4.39 | 5.0 |
| DC9045⁵ | 11.0 | 11.0 | 5.5 |
| Isopropyl lauroyl | 7.32 | 7.32 | — |
| sarcosinate | | | |
| Laureth-4 | 0.2 | 0.2 | 0.2 |
| Particles: | | | |
| KSP 102⁶ | 11.0 | 11.0 | |
| KSP 105⁷ | 11.0 | 11.0 | |
| Dry Flo TS⁸ | — | — | 20.0 |
| Total: | 100% | 100% | 100% |

¹1,2-hexanediol and caprylyl glycol, from Symrise
²Iodopropynyl butylcarbamate, PEG-4 laurate, PEG-4 dilaurate, and polyethylene glycol, from Lonza
³DMDM Hydantoin, Butane-1,3-diol, iodopropynyl butylcarbamate, water, from Lonza
⁴Sodium acrylate/sodium acryloyldimethyl taurate copolymer, isohexadecane, and polysorbate 60, from Seppic
⁵Cyclopentasiloxane and dimethicone crosspolymer, from Dow Corning
⁶Vinyl dimethicone/methicone silsesquioxane crosspolymer, from Shin Etsu
⁷Vinyl dimethicone/methicone silsesquioxane crosspolymer, from Shin Etsu
⁸Tapioca and polymethylsilsesquioxane, from Akzo Nobel

All data provided below is measured according to the VAT test described above and is statistically significant to an 80% confidence level (at alpha=0.20, Tukey tests).

### Comparative Example A and Invention Examples 3+11+21

### Impact of Layers

Examples A, 3, 11 and 21 are prepared using the same process used for the examples above.

Composition A is an all-in-one, or single step, formulation that contains the same glycerin and silicone elastomer powder systems as the three layer system consisting of composition 3 (the first composition), followed by composition 11 (the second composition), followed by composition 21 (the third composition). Note also that for the all-in-one versus three step composition comparisons, few very minor formula adjustments were made to ensure adequate stability and physical properties. However, these minor formula adjustments are not expected to significantly impact the optical benefit of these formulations.

The all-in-one and the three step compositions were placed in a VAT to compare their effectiveness at reducing the appearance of various facial attributes. The VAT data below compares the performance of the same glycerin (25%) and powder (11% KSP 102 plus 11% KSP 105) systems in a one layer (all-in-one) and a three layer product, with glycerin being in the bottom layer (against the skin) and powder being in the outermost layer (farthest from the skin surface) for the three layer system. As can be seen, the three layer product delivered significantly greater Cheek Fine Lines and Bumpy Surface benefits with directionally greater pore benefits than the one layer (all-in-one) formulation.

| | One Layer | |
|---|---|---|
| | (All-in-One) - | Three Layers - |
| | Ex A | Ex 3 + 11 + 21 |
| Cheek Fine Lines | 0.18 b | 0.37 a |
| Bumpy Surface | 0.09 b | 0.37 a |
| Pores | 0.09 a | 0.25 a |

* Products with the same letter are not significantly different from each other at alpha = 0.05 (Tukey tests)

### Comparative Examples A and B and Invention Examples 3+11+21

### Impact of Glycerin Levels

Three different formulations were compared, all with the same powder system (11% KSP 102 plus 11% KSP 105): one layer (all-in-one) systems containing either 5% (Example B) or 25% (Example A) glycerin, and a three layer system of the present invention containing 25% glycerin (Example 3+Example 11+Example 21).

Compositions A, B and 3+11+21 are placed in a VAT to compare their effectiveness at reducing the appearance of various facial attributes. As can be seen from the results of this testing, summarized in the table below, increasing the level of glycerin from 5% to 25% significantly reduced the optical benefit provided by the elastomer particles and non-volatile silicone oil combination in these products.

Larger VAT scores for cheek fine lines, bumpy surface, and pores correspond to bigger visible reductions in the appearance of these attributes. The results summarized below demonstrate the negative effects of combining a high moisturizing composition with a high particulate composition in an “all-in-one” composition, and the benefits of the three layer compositions of the present invention. As can be seen, increasing glycerin from 5% to 25% in a one layer (all-in-one) system resulted in significant and directional (pores) loss in optical benefits. Further, as discussed above, for the two 25% glycerin systems, separating the glycerin and powders into a three layer system provided significantly and directionally (pores) greater optical benefits than if the glycerin and powders were combined into one layer (all-in-one product). Finally, when the glycerin level is very low (5%) in a one layer (all-in-one) system, the optical benefits of a one layer system match those of a three layer system with high glycerin. Thus, an optical benefit of layering is the ability to deliver high optical benefits together with high levels of glycerin (for moisturization/skin health).

| | 5% Glycerin | 25% Glycerin | 25% Glycerin |
|---|---|---|---|
| | All-In-One- | All-In-One - | Three Layer Ex |
| | Ex B | Ex A | Examples 3 + 11 + 21 |
| Cheek Fine Lines | 0.40 a | 0.18 b | 0.37 a |
| Bumpy Surface | 0.29 a | 0.09 b | 0.37 a |
| Pores | 0.27 ab | 0.09 b | 0.25 ab |

* Products with the same letter are not significantly different from each other at alpha = 0.05 (Tukey tests)

### EXAMPLES

### Consumer Use Data

Consumer Test 1—

One and three layer systems were placed in a four week consumer test. Products were placed with approximately 50 panelists. Each product was placed in blind, white pumps. Consumers were asked to use the products ad lib for four weeks, and then rate the products at the conclusion of the four weeks. The two products examined in the data below consist of a one layer (all-in-one) system (Example A), as well as a three layer system (Examples 3+11+21) of the present invention. Both products contained 25% glycerin and 11% KSP-102 and 11% KSP-105. The data below shows that the panelists saw a significantly greater change in the appearance and feel of skin when using the three step system vs. the one step system.

The following scale was used to generate the consumer data. Panelists were asked to rate the products' effectiveness at improving the appearance of their skin on a five point scale (Improved A Lot=+2, Improved A Little=+1, No Change=0, Worsened A Little=−1, Worsened A Lot=−2). The data below shows the average numerical response on the −2 to +2 scale. The same scale was used for feel.

| | One Layer | |
|---|---|---|
| | (All-In-One) | Three Layers |
| | 22% Powder - | 22% Powder - |
| | Ex A | Ex 3 + 11 + 21 |
| Difference Noticed in Overall | 0.61 b | 1.02 a |
| Appearance of Skin | | |
| (degree of improvement on | | |
| a −2 to +2 point scale) | | |
| Difference Noticed in Overall | 1.11 b | 1.35 a |
| Feel of Skin | | |
| (degree of improvement on | | |
| a −2 to +2 point scale) | | |

* Products with the same letter are not significantly different from each other at alpha = 0.05 (Tukey tests)

Consumer Test 2—

One and three layer systems were also placed in a 2 week consumer test. Products were placed with approximately 80 panelists. Each product was placed in blind, white pumps. Consumers were asked to use the products ad lib for two weeks, and then rate the products at the conclusion of the two weeks. The data table below examined data for three products. The first is a one layer (all-in-one) system with 15% glycerin and 20% Dry Flo TS (Example C). The second is a three layer system of the present invention with 15% glycerin and 20% Dry Flo TS (Examples 2+12+22). The third product is a three layer system of the present invention (Examples 1+13+23) with 25% glycerin, 11% KSP-102 and 11% KSP-105 in the third layer and significant levels of active ingredients in the first layer (i.e., Example 1). Note that for the all-in-one versus three step composition comparisons, few very minor formula adjustments were made to ensure adequate stability and physical properties. However, these minor formula adjustments are not expected to significantly impact the optical benefit of these formulations.

The data below show that the panelists saw a significantly greater change in the appearance and feel of skin when using the three step system vs. the one step system. Note also that the difference between compositions comprising Examples 2+12+22 compared to compositions comprising Examples 1+13+23 is that the latter contains additional skin active ingredients (Sepiwhite MWH, Biomyox, Palestrina, Olivem 360 and Hexyldecanol). The data below confirm that the inclusion of various skin care actives does not impair the benefits provided by the present invention.

Note the following scale was used to generate the consumer data. Panelists were asked to rate the products' effectiveness at improving the appearance of their skin on a five point scale (Improved A Lot=+2, Improved A Little=+1, No Change=0, Worsened A Little=−1, Worsened A Lot=−2). The data below shows the percentage of panelists that rated the product in the top box (Improved A Lot). The same scale was used for feel.

| | One Layer | | Three Layers |
|---|---|---|---|
| | (All-In-One) | Three Layers | 11% KSP-105 |
| | 20% Dry Flo TS - | 20% Dry Flo TS | 11% KSP-102 |
| | Ex C | Ex 2 + 12 + 22 | Ex 1 + 13 + 23 |
| Overall Appear- | 8 b | 19 a | 18 a |
| ance - Im- | | | |
| proved a lot | | | |
| Overall Feel - | 13 b | 29 a | 42 a |
| Improved a lot | | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of improving the look or feel of human skin comprising the following steps:
a. applying to the skin a first layer formed using a first composition comprising a humectant;
b. thereafter applying a second layer formed using a second composition comprising a non-volatile oil; and
c. thereafter applying a third layer formed using a third composition comprising from about 10% to about 30%, by weight of the third composition, of substantially spherical particles selected from the group consisting of coated starch, uncoated starch, coated starch derivatives, uncoated starch derivatives, coated crosslinked starch, uncoated crosslink starch, coated silicone elastomer particles, uncoated silicone elastomer particles, and combinations thereof.

2. The method of claim 1, wherein the first composition comprises from about 5% to about 30%, by weight of the first composition, of at least one humectant.

3. The method of claim 2, wherein the humectant is glycerin.

4. The method of claim 1, wherein the first composition is water based and is in the form of an aqueous solution, an aqueous gel, an aqueous dispersion, an oil-in-water emulsion, or a water-in-oil-in-water emulsion.

5. The method of claim 1, wherein the second composition comprises a non-volatile silicone oil.

6. The method of claim 5, wherein the second composition comprises at least about 70%, by weight of the non-volatile oil, of the non-volatile silicone oil.

7. The method of claim 6, wherein the first and second compositions each have a contrast ratio of less than about 20.

8. The method of claim 5, wherein the second composition comprises dimethicone.

9. The method of claim 1, wherein the first, the second and the third compositions each comprises less than about 4%, by weight of the third composition, of mica, talc, and other substantially non-spherical particles.

10. The method of claim 1, wherein the second composition comprises at least about 50%, by weight of the nonvolatile oil, of a UV agent.

11. The method of claim 1, wherein the third composition comprises less than about 1%, by weight of the third composition, of TiO₂, iron oxide particles and other pigment particles.

12. The method of claim 1, wherein the first composition comprises at least about 1%, by weight of the first composition, of one or more skin care actives selected from the group consisting of sugar amines, vitamins, oil control agents, photosterols, hexamidine compounds, tightening agents, anti-wrinkle actives, anti-atrophy actives, flavonoids, N-acyl amino acid compounds, retinoids, peptides, anti-cellulite agents, desquamation actives, anti-acne actives, anti-oxidants, radical scavengers, anti-inflammatory agents, tanning actives, skin lightening agents, botanical extracts, antimicrobial actives, antifungal actives, antibacterial actives, sensates, and combinations thereof.

13. The method of claim 1, wherein the substantially spherical particles in the third composition have an average diameter of from about 1 μm to about 40 μm.

14. The method of claim 1, wherein the weight ratio of non-volatile oil in the second composition to substantially spherical particulate material in the third composition is from about 1:10 to about 3:2.

15. The method of claim 14, wherein the weight ratio of non-volatile oil in the second composition to substantially spherical particulate material in the third composition is from about 1:4 to about 1:1.

16. (canceled)

17. (canceled)

18. (canceled)

19. (canceled)

20. (canceled)
